(19) United States
(12) Patent Application Publication
(10) Pub. No.: US 2006/0129242 A1
(43) Pub. Date: Jun. 15, 2006

(54) Pseudo arthrosis device

(76) Inventors: Brian Bergeron, Austin, TX (US); Reginald J. Davis, Cockeysville, MD (US); Michael Funk, North Bend, WA (US)

Correspondence Address: ROBERT DEBERARDINE;ABBOTT LABORATORIES, 100 ABBOTT PARK ROAD, DEPT. 377/AP6A, ABBOTT PARK, IL, 60064-6008, US

(21) Appl. No.: 11/147,526

(22) Filed: Jun. 8, 2005

Related U.S. Application Data
(63) Continuation-in-part of application No. 10/331,191, filed on Dec. 27, 2002.
Application No. 10/331,191 is a continuation-in-part of application No. 10/035,052, filed on Dec. 28, 2001, now Pat. No. 6,736,850.

(51) Int. Cl. A61F 2/44

(52) U.S. Cl. 623/17.16

(57) ABSTRACT

A pseudo arthrosis device may be placed in a disc space to provide support for adjacent vertebrae. The device may have an enclosure formed from a flexible, permeable material. A core may be packed in the enclosure. The core may be compressible and/or flexible. In certain embodiments, the core may include a plurality of elongated members. In other embodiments, the core may include ridges or grooves. The enclosure may be placed in an intervertebral space between the adjacent vertebrae such that the core is disposed between the adjacent vertebrae. The pseudo arthrosis device may include a tab designed to couple the enclosure to an adjacent vertebrae.

### PRIORITY CLAIM

This application is a continuation-in-part of, and incorporates by reference, U.S. patent application Ser. No. 10/331,191, entitled “PSEUDO ARTHROSIS DEVICE,” filed on Dec. 27, 2002, which is a continuation-in-part of U.S. patent application Ser, No. 10/035,052, entitled “PSEUDO ARTHROSIS DEVICE,” filed on Dec. 28, 2001, now U.S. Pat. No. 6,736,850.

### BACKGROUND

1. Field of Invention

The present invention generally relates to spinal implants for alleviating problems in human spines. More particularly, one aspect of the invention relates to an pseudo arthrosis device (or intradiscal stabilization device) that replaces a damaged spinal disc, and another aspect of the invention relates to artificial anterior longitudinal ligaments (ALLs).

2. Description of Related Art

A spinal disc is a shock-absorbing structure located in a space between two adjacent vertebrae in a spine. A spinal disc may deteriorate due to trauma, disease, and/or aging. A deteriorated spinal disc may have diminished shock-absorbing capacity. A deteriorated disc may allow adjacent vertebrae to contact each other. Contact of adjacent vertebrae may result in wear and tear of the vertebrae. Wear and tear of the vertebrae may result in pain (e.g., neck and/or back pain).

Non-surgical treatments to reduce neck and/or back pain may include rest, heat, medication, physical therapy, and chiropractic manipulation. Non-surgical treatments may be ineffective for some patients.

Surgical treatment of a deteriorated spinal disc may include spinal fusion. A spinal fusion treatment may not be successful in some patients. When successful, spinal fusion treatments may result in stiffness and decreased mobility of the patient. Spinal fusion may cause stress on the spine at adjacent vertebral levels. The stress on the spine may create new spinal problems. New spinal problems may result in additional neck and/or back pain.

Surgical alternatives to spinal fusion may include spinal disc replacement. U.S. Pat. No. 3,567,728 to Stubstad et al.; U.S. Pat. No. 5,071,437 to Steffee; U.S. Pat. No. 5,320,644 to Baumgartner; U.S. Pat. No. 5,522,899 to Michelson; U.S. Pat. No. 5,961,554 to Janson et al.; U.S. Pat. No. 5,976,186 to Bao et al.; U.S. Pat. No. 6,162,252 to Kuras et al.; U.S. Pat. No. 6,206,924 to Timm; U.S. Pat. No. 6,214,049 to Gayer et al.; and U.S. Pat No. 6,093,205 to McLeod et al., each of which is incorporated by reference as if fully set forth herein, describe devices for surgical treatment of deteriorated and/or damaged spinal discs.

### SUMMARY

A pseudo arthrosis device may be a relatively inexpensive, simple device that is easily inserted into the spine to replace a damaged or degenerated disc. The pseudo arthrosis device may be compatible with the human body. In some embodiments, a pseudo arthrosis device may serve as a matrix or scaffold to support growth of body fibers in a patient. Growth of the patient's body fibers may incorporate the device into the patient. Incorporation of the device into the patient may allow the patient's body to repair itself.

In one embodiment, an implant device includes an enclosure that includes a core packed within the enclosure, and a tab. The core includes a plurality of elongated members. The tab is coupled to the enclosure for attaching the implant to a vertebra.

In another embodiment, an implant device includes an enclosure that includes a core packed within the enclosure, and a tab. The core includes at least one groove on a surface of the core. The tab is coupled to the enclosure for attaching the implant device to a vertebra.

Another aspect of the invention relates to artificial anterior longitudinal ligaments. Natural anterior longitudinal ligaments may become degenerate or become damaged in the course of an operation. The artificial anterior longitudinal ligament can provide stability to the spine in such situations. In one embodiment, an artificial ligament implant includes a tab that has elasticity. The tab includes a pair of openings that are configured to couple the tab to a pair of vertebrae. The tab serves as an artificial longitudinal ligament.

### BRIEF DESCRIPTION OF THE DRAWINGS

FIG. 1 is a perspective view of a pseudo arthrosis device.

FIG. 2 is a side view of an elongated member.

FIG. 3 is a cross-sectional view taken substantially along plane 3-3 of FIG. 2.

FIG. 4 is a side view of a pseudo arthrosis device.

FIG. 5 is a back view of a pseudo arthrosis device.

FIG. 6 is a cross-sectional view taken substantially along plane 6-6 of FIG. 4.

FIG. 7 is a cross-sectional view taken substantially along plane 7-7 of FIG. 5.

FIG. 8 is a perspective view of a pseudo arthrosis device with a pliable retainer in an enclosure.

FIG. 9 depicts a portion of a spinal column with a deteriorated disc.

FIG. 10 depicts a pseudo arthrosis device positioned for insertion into a disc space.

FIG. 11 depicts insertion of a pseudo arthrosis device into a disc space.

FIG. 12 depicts a pseudo arthrosis device positioned in a disc space.

FIG. 13 depicts a portion of a spine showing compression of an implanted pseudo arthrosis device when a patient bends backwards.

FIG. 14 depicts a portion of a spine showing compression of an implanted pseudo arthrosis device when a patient bends forwards.

FIG. 15 depicts a portion of a spine showing compression of a pseudo arthrosis device when a patient leans laterally to the right.

FIG. 16 depicts a portion of a spine showing compression of a pseudo arthrosis device when a patient leans laterally to the left.

FIG. 17 is an enlarged view showing placement of a pseudo arthrosis device in a disc space.

FIG. 18 is a cross-sectional view taken substantially along line 18-18 of FIG. 17 showing fibrous ingrowth.

FIG. 19 is an enlarged view of an elongated member showing fibrous ingrowth.

FIG. 20 is a perspective view of a pseudo arthrosis device with an enclosure containing a core.

FIG. 21 is a side view of a pseudo arthrosis device.

FIG. 22 is a perspective view of a pseudo arthrosis device that includes a tab and a core.

FIG. 23 is a perspective view of a pseudo arthrosis device with openings fitted with grommets.

FIGS. 24A-24H are perspective views of cores for use in pseudo arthrosis devices.

FIGS. 25-26 show pseudo arthrosis devices positioned in a patient's spine.

FIGS. 27-28 show a plurality of pseudo arthrosis devices in a multi-level implant application.

FIG. 29 is a perspective view of an artificial anterior longitudinal ligament.

FIG. 30 is a side view of an artificial anterior longitudinal ligament.

FIG. 31 is an artificial anterior longitudinal ligament positioned in a patient's body.

FIG. 32 shows a plurality of artificial anterior longitudinal ligaments in a multi-level implant application.

FIG. 33 is a perspective view of an alternative embodiment of an artificial anterior longitudinal ligament.

FIG. 34 shows the artificial anterior longitudinal ligament of FIG. 33 positioned in a patient's body.

FIG. 35 shows a plurality of the artificial anterior longitudinal ligaments of FIG. 33 in a multi-level implant application.

FIG. 36 shows an alternative application of the artificial anterior longitudinal ligament of FIG. 33.

FIG. 37 shows an alternative application of the artificial anterior longitudinal ligament of FIG. 33 in a multi-level implant.

While the invention may be susceptible to various modifications and alternative forms, specific embodiments thereof are shown by way of example in the drawings and will herein be described in detail. The drawings may not be to scale. It should be understood that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF EMBODIMENTS

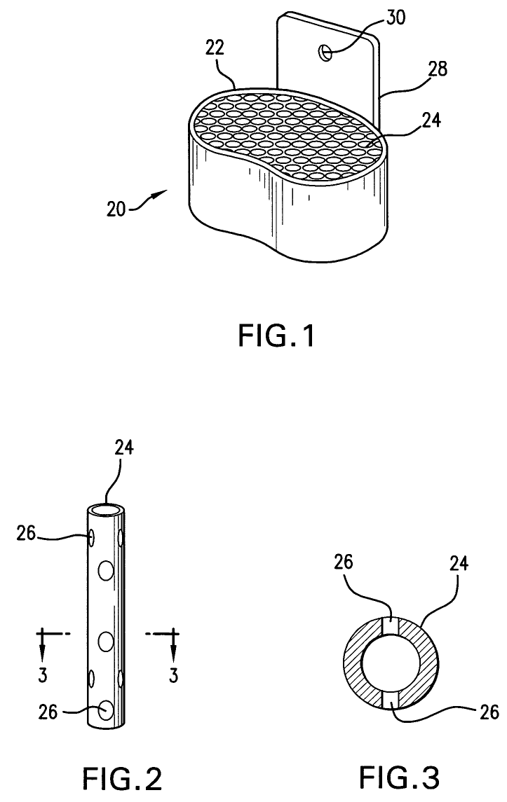

FIG. 1 depicts pseudo arthrosis device 20 with enclosure 22. Enclosure 22 may be formed from a non-allergenic, biocompatible material. The material may be flexible and/or permeable. The material may include, but is not limited to, synthetic fabrics such as Dacron®, Proline Meshg®, and Goretex®. Enclosure 22 may have a cross-sectional shape corresponding to a cross-sectional shape of an intervertebral space between two adjacent vertebrae in a human spine. Enclosure 22 may be in the form of a tube, a sleeve, an envelope, or other suitable configuration.

As shown in FIG. 1, enclosure 22 may include a plurality of elongated members 24. Elongated members 24 may be compressible and/or flexible. Elongated members 24 may be made of biocompatible materials including, but not limited to, polyethylene, high density polyethylene, ultra high density polyethylene, and various fluorinated polymers. Advantageously, a pseudo arthrosis device including a plurality of elongated members 24 may offer more adaptability, compressibility, and/or flexibility than a device formed of a single piece of substantially the same material.

Longitudinal placement of elongated members 24 in enclosure 22 may allow pseudo arthrosis device 20 to function as a compressible, flexible body. Since the compressibility and flexibility arise from a plurality of elongated members 24, pseudo arthrosis device 20 may provide greater local adaptation to stress between vertebrae than a single deformable piece of material. Flexibility of elongated members 24 and enclosure 22 may allow movement in non-longitudinal directions (e.g., bending and rotation) relative to axes of elongated members 24.

In some embodiments, elongated members 24 may be solid rods. In certain embodiments, elongated members 24 may be hollow tubes. For example, an outer diameter of hollow elongated member 24 may range from about 0.5 mm to about 3.0 mm. An inner diameter of hollow elongated member 24 may range from about 0.25 mm to about 2.0 mm. In an embodiment, elongated member 24 may have a wall thickness of about 1 mm.

FIG. 2 depicts hollow elongated member 24 with a plurality of spaced-apart perforations 26. Flexibility of elongated member 24 may be a function of wall thickness of the elongated member and number and size of perforations 26. Perforations 26 may increase flexibility of solid as well as hollow elongated members 24. A length of elongated member 24 may be chosen to achieve desired intervertebral spacing and/or desired flexibility. FIG. 3 depicts a cross-sectional view of hollow elongated member 24 with perforations 26 taken substantially along plane 3-3 of FIG. 2.

As shown in FIG. 1, pseudo arthrosis device 20 may include at least one tab 28. Tab 28 may be of any suitable size, shape, or configuration to couple pseudo arthrosis device 20 to a vertebra. In some embodiments, tab 28 may be an integral part of pseudo arthrosis device 20. In other embodiments, tab 28 may be coupled to enclosure 22. In some embodiments, tab 28 may extend substantially perpendicularly upward from enclosure 22. In other embodiments, tab 28 may extend substantially perpendicularly downward from enclosure 22. In certain embodiments, tab 28 may extend substantially perpendicularly upward and downward from enclosure 22. Alternatively, a first tab 28 may extend substantially perpendicularly upward from enclosure 22 and a second tab 28 may extend substantially perpendicularly downward from enclosure 22. In certain embodiments, enclosure 22 and tab 28 may be made of substantially the same material.

Tab 28 may include at least one opening 30. Opening 30 may be reinforced with a grommet. A grommet used to reinforce opening 30 may be made of titanium or any other durable biocompatible material. In an embodiment, pseudo arthrosis device 20 may be secured to a vertebra by a connector or fastener inserted through opening 30. In certain embodiments, a connector may be a threaded screw. Alternatively, tab 28 may be connected to a vertebra by methods including the use of, but not limited to the use of, sutures, staples, barbs, and/or adhesive.

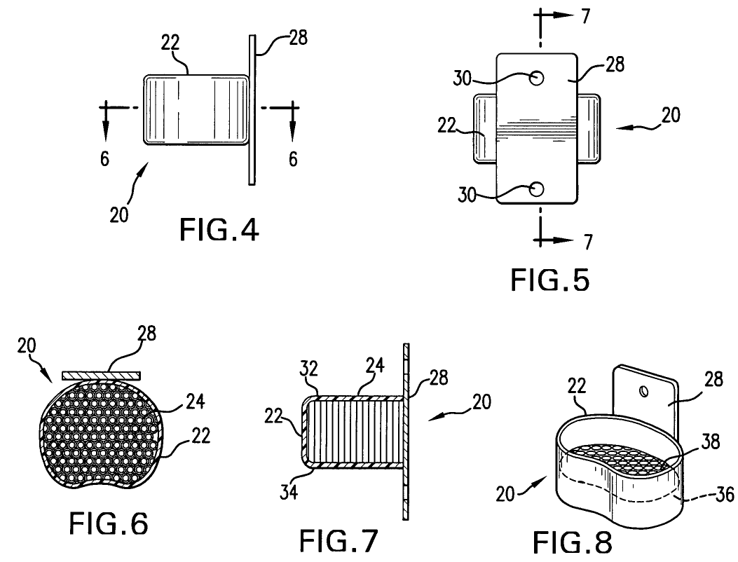

FIG. 4 depicts a side view of pseudo arthrosis device 20 with enclosure 22 and tab 28. FIG. 5 depicts a back view of pseudo arthrosis device 20 with enclosure 22, tab 28, and openings 30.

FIG. 6 depicts a cross-sectional view of pseudo arthrosis device 20 taken substantially along plane 6-6 of FIG. 4. FIG. 7 depicts a cross-sectional view of pseudo arthrosis device 20 taken substantially along plane 7-7 of FIG. 5. FIGS. 6 and 7 depict a plurality of elongated members 24 placed longitudinally in enclosure 22 with coupled tab 28. In the embodiment shown in FIG. 7, enclosure 22 has top 32 and bottom 34 to promote retention of elongated members 24 in the enclosure. In some embodiments, top 32 and/or bottom 34 may be made of bioabsorbable material (e.g., Surgicel).

In some embodiments, such as the embodiment shown in FIG. 8, pliable retainer 36 may be located transversely in enclosure 22 of pseudo arthrosis device 20. Pliable retainer 36 may have a plurality of spaced-apart holes 38. Elongated members may be positioned securely in holes 38 (e.g., with a friction fit). Elongated members in holes 38 may be retained in an upright packed condition (oriented longitudinally) when pseudo arthrosis device 20 is inserted into a disc space between adjacent vertebrae.

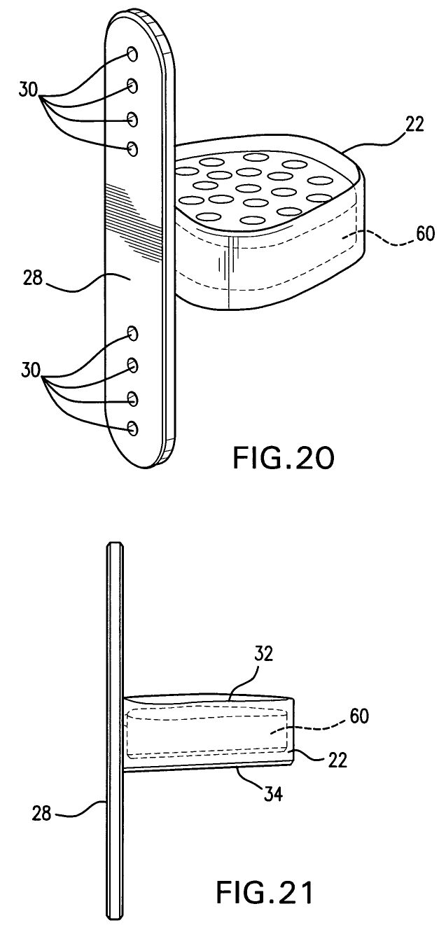

In other embodiments, such as the embodiment shown in FIG. 20, rather than elongated members 24, a core 60 may be used in device 20. Core 60 is made of viscoelastic material, such as flexible polymers, hydrogels, collagen, and the like. Generally, core 60 may be made of biocompatible materials. Core 60 provides adaptability, compressibility, pliability and/or flexibility as an artificial disc when inserted in the intervertebral space in a spine. Core 60 may have a plurality of elongated members, columns, walls, bore holes, grooves, and/or dimples, as described below in detail.

FIG. 21 shows a side view of an illustrative embodiment of a pseudo arthrosis device 20. In this embodiment, enclosure 22 forms a jacket around core 60 (core 60 packed within enclosure 22). Similar to the configuration shown in FIG. 7, enclosure 22 has top 32 and bottom 34 to promote retention of core 60 in the enclosure.

Tab 28 and enclosure 22 may generally have the configurations and be made of the same materials described above. In the illustrative embodiment shown in FIG. 20, tab 28 has a first portion or tab that extends upward from enclosure 22, and a second portion or tab that extends downward from enclosure 22. Furthermore, tab 28 includes a plurality of openings 30 on each of the portions that extend upward and downward from enclosure 22.

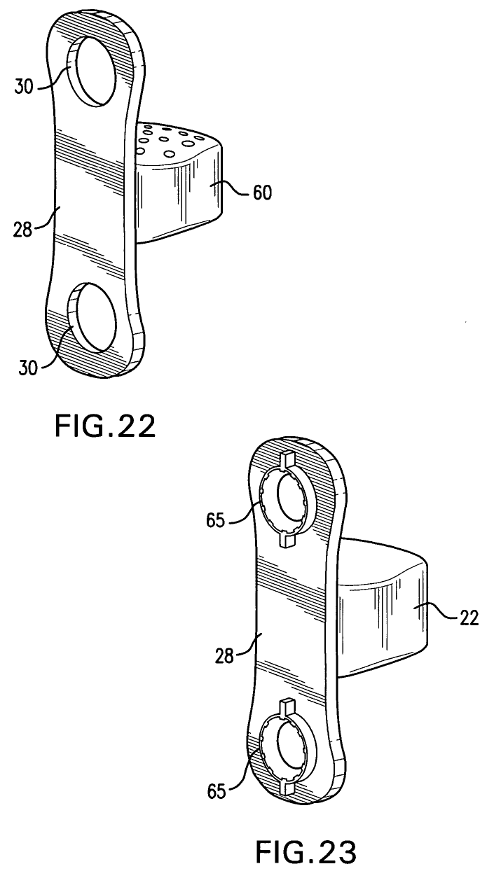

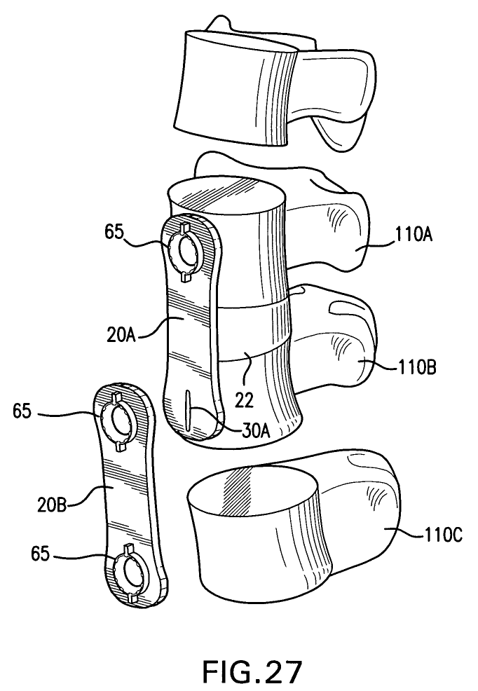

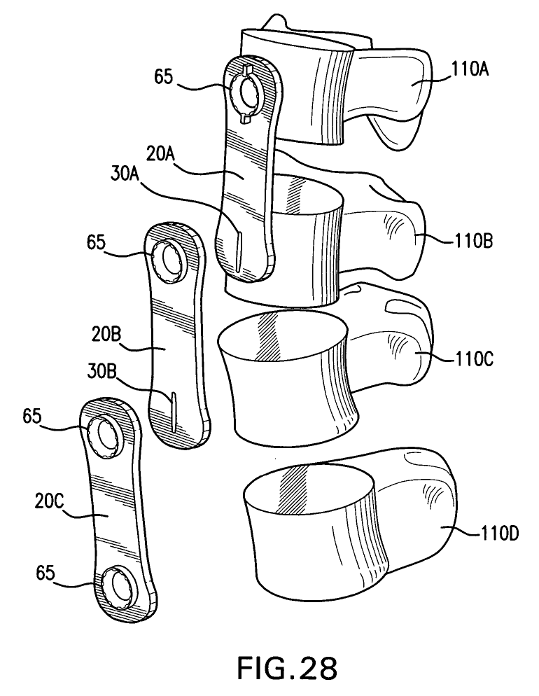

A variety of numbers, shapes, and configurations of openings 30 may be used, as desired. For example, the embodiment shown in FIG. 22 includes a substantially round opening 30 in each of the upward and downward portions of tab 28. As another example, the embodiment shown in FIG. 23 uses grommets 65 in each of the openings in the tab. As yet another example, openings 30 may have an elongated shape, similar to the shape of a button hole (see FIGS. 27 and 28 and their corresponding descriptions).

In some embodiments, as shown for example in FIG. 22, core 60 is coupled to tab 28 without the use of an enclosure. In other embodiments, tab 28 and enclosure 22 may have a one-piece configuration or, alternatively, may be separate pieces coupled together (for example, sewn together, glued together, stapled together, and the like).

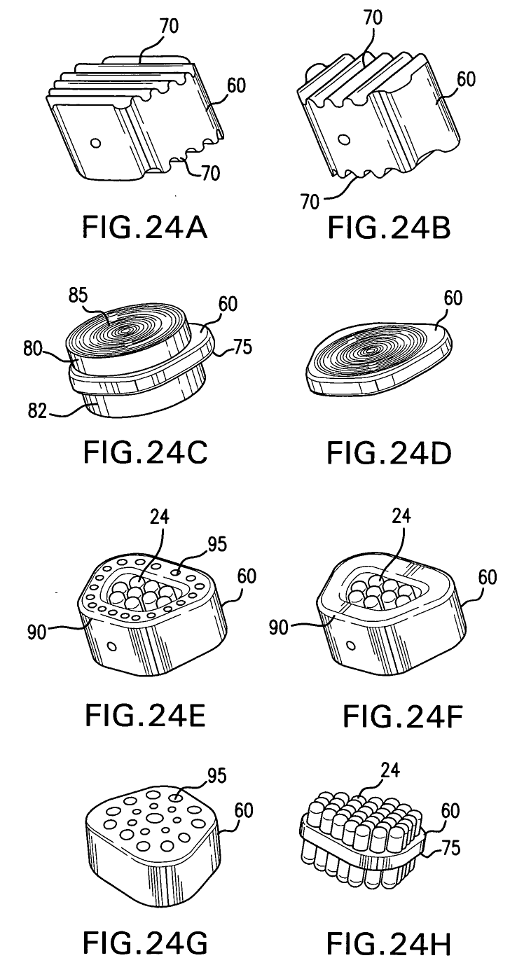

FIGS. 24A-24H show illustrative embodiments of core 60 for use in pseudo arthrosis devices. In the embodiment of FIG. 24A, core 60 has a plurality of ridges or grooves 70 on its top and bottom surfaces. Ridges 70 have a lateral direction (side-to-side, when implanted in the patient's body). The embodiment of FIG. 24B also includes a plurality of ridges or grooves 70 on its top and bottom surfaces. Ridges 70 in the core in FIG. 24B, however, have a front-to-back (anterior-to-posterior, when implanted in the patient's body) direction. In yet another configuration (not shown), the ridges on the top and bottom surfaces of core 60 may have differing directions, a mixture of directions, and/or other directions (e.g., diagonal) as desired.

Core 60 in FIG. 24C has a plate 75 that couples to an upper portion 80 and lower portion 82 of core 60. The top surface of core 60 (i.e., the top surface of upper portion 80) has a plurality of grooves 85 formed as co-centric circles. The bottom surface of core 60 may have a similar or different arrangement of grooves. Grooves 85 help to promote ingrowth of tissue into core 60.

Core 60 in FIG. 24D has a plurality of grooves 85 on its top surface. The plurality of grooves 85 may be co-centric circles. The bottom surface of core 60 may have a similar or different arrangement of grooves. Similar to the core in FIG. 24C, grooves 85 help to promote ingrowth of tissue into core 60.

Note that grooves 85 in FIGS. 24C and 24D may have other features or configurations, such as one or more spirals, as desired. Furthermore, top and bottom surfaces of core 60 may have different features or configurations of grooves 85, as desired (for example, circles on one surface of core 60, and a spiral on another surface).

The cores in FIGS. 24E and 24F include a plurality of elongated members 24 arranged within an outer portion or shell 90. Outer portion 90 generally surrounds elongated members 24. Elongated members 24 may be integral to core 60, or may be constructed by using a pliable retaining plate (not shown explicitly), similar to the embodiment of FIG. 8. Core 60 in FIG. 24E has the additional feature of a plurality of dimples or indentations 95 on the top surface of outer portion 90. Indentations 95 promote ingrowth of tissue and help to stabilize the patient's spine. The bottom surface of outer portion 90 may have a similar or different arrangement of indentations, as desired. Note that indentations 95 may have a variety of shapes (round or annular, triangular, rectangular, etc.), configurations (e.g., depth), and arrangements, as desired.

Core 60 in FIG. 24G has a plurality of dimples or indentations 95 arranged on its top surface. Indentations 95 help to promote ingrowth of tissue. The bottom surface of outer portion 90 may have a similar or different arrangement of indentations, as desired. Indentations 95 may have a variety of shapes (round or annular, triangular, rectangular, etc.), configurations (e.g., depth), and arrangements, as desired.

The core in FIG. 24H includes a plate 75 and a plurality of elongated members 24. Elongated members 24 may be integral to core 60, or may be constructed by using a pliable retaining plate (not shown explicitly), similar to the embodiment of FIG. 8. Elongated members 24 may have a variety of shapes, configurations, and arrangements, as described above.

Note that a core (or cores in multi-level applications, as described below) configuration may be used that allows certain forces in the spine, while limiting or tending to limit other forces. For example, the cores shown in FIGS. 24A, 24B, and 24H tend to allow translational movements of the affected vertebrae. The cores shown in FIGS. 24C-24G, however, tend to limit translational movements and provide a more rigid support to the spine. Furthermore, generally, the number, shape, size, type, and arrangement of the various physical attributes of the cores (e.g., the number of ridges, pores, dimples, elongated members, etc.) may be varied to suit a particular application, as desired.

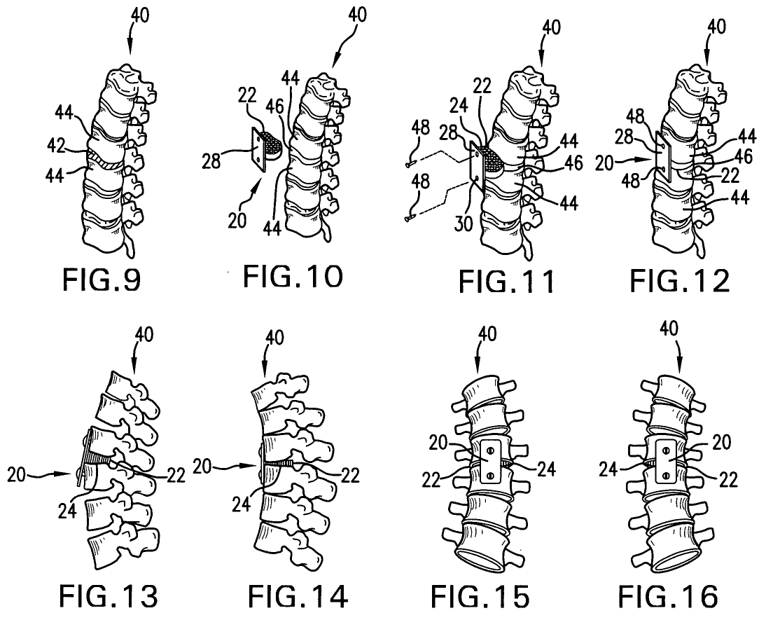

FIGS. 9-12 depict a method of implanting a pseudo arthrosis device. FIG. 9 depicts a portion of a spinal column 40 with deteriorated disc 42 between adjacent vertebrae 44. Deteriorated disc 42 may be removed from spinal column 40 to form disc space 46, as shown in FIG. 10.

FIG. 10 depicts pseudo arthrosis device 20 with enclosure 22 and tab 28 positioned for insertion into disc space 46 between adjacent vertebrae 44. As shown in FIG. 11, elongated members 24 in enclosure 22 are disposed longitudinally between adjacent vertebrae 44 in disc space 46. In an embodiment, tab 28 may be coupled to adjacent vertebrae 44 with connectors 48. Tab 28 may be sized to secure enclosure 22 to adjacent vertebrae 44 with minimal limitation of vertebral movement. FIG. 12 depicts pseudo arthrosis device 20 implanted into spinal column 40. In an embodiment, tab 28 and connectors 48 may secure enclosure 22 of pseudo arthrosis device 20 in disc space 46 between adjacent vertebrae 44.

FIGS. 13-16 show flexion and compression of elongated members 24 following implantation of pseudo arthrosis device 20 in spinal column 40 of a patient. As the patient bends backwards (FIG. 13) or forwards (FIG. 14), elongated members 24 proximal to the bend of the spine are compressed while the elongated members distal to the bend of the spine are fully extended. FIGS. 15 and 16 depict compression of elongated members 24 when a patient leans laterally right and left, respectively. Compression and extension of elongated members 24 may cushion vertebrae of spinal column 40, thereby simulating natural disc function. In some patients, pseudo arthrosis device 20 may reduce wear and tear on vertebrae of a spinal column and thus relieve spinal pain.

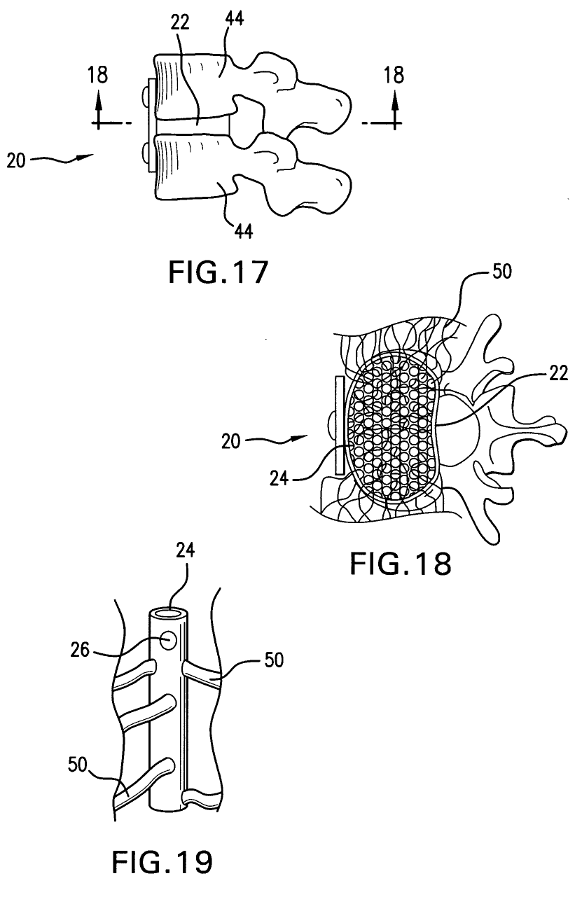

FIG. 17 depicts pseudo arthrosis device 20 after implantation in a disc space between adjacent vertebrae 44. FIG. 18 depicts a cross-sectional view of pseudo arthrosis device 20 taken substantially along line 18-18 in FIG. 17. As shown in FIG. 18, fibers 50 of a patient may penetrate enclosure 22. Fibers 50 that penetrate enclosure 22 may promote fibrous tissue growth in spaces between elongated members 24. Normal movement of the patient and flexibility of pseudo arthrosis device 20 may inhibit conversion of fibrous tissue growth in enclosure 22 into bone.

FIG. 19 depicts growth of fibers 50 through perforations 26 in elongated member 24. In an embodiment, fibers 50 may grow longitudinally through hollow elongated member 24. Over time, pseudo arthrosis device 20 may be incorporated into scar tissue formed from fibrous ingrowth of a patient. In certain embodiments, pseudo arthrosis device 20 may advantageously function as a scaffold or matrix to promote natural body repair by fibrous ingrowth.

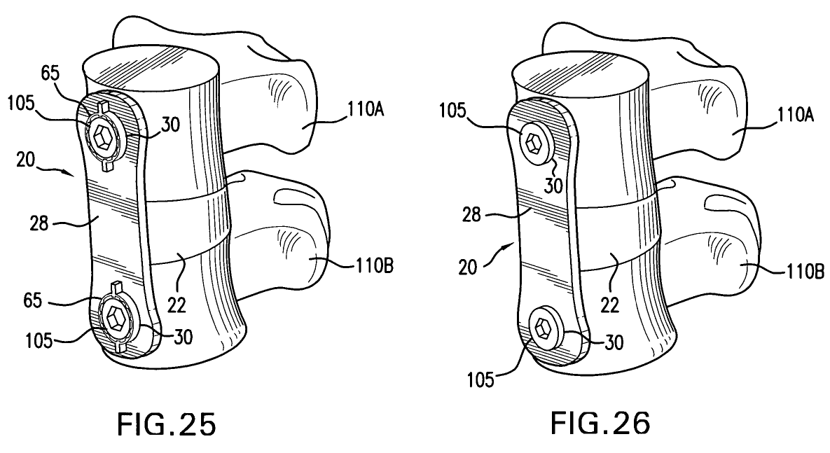

FIGS. 25-28 show illustrative embodiments of pseudo arthrosis device 20, including core 60, implanted within a spine. FIGS. 25-26 show implants for a single level, whereas FIGS. 27-28 illustrate implants for multiple levels.Note that the figures show two-level or three-level applications as illustrative examples, and that the disclosed implants may be used generally in multi-level applications involving different numbers of levels.

Referring to FIG. 25, pseudo arthrosis device 20 couples to vertebrae 110A and 110B. In the embodiment shown, pseudo arthrosis device 20 has openings 30 fitted with grommets 65. A pair of screws 105 fasten the upper and lower parts of tab 28 to vertebrae 110A and 110B, respectively. In the embodiment shown in FIG. 26, screws 105 (or other suitable fastener) fasten the upper and lower parts of tab 28 to vertebrae 110A and 110B, respectively, without the use of grommets.

FIG. 27 shows two pseudo arthrosis devices, 20A and 20B, in a multi-level implant, involving vertebrae 110A-110C. Note that one of the openings for pseudo arthrosis device 20A may overlap with one of the openings for pseudo arthrosis device 20. In such a situation, one of the pseudo arthrosis devices may use an opening without a grommet 65, such as opening 30A of pseudo arthrosis device 20A. Opening 30A provides a lower profile for the two overlapping openings of pseudo arthrosis devices 20A and 20B. Opening 30A may have a variety of desired shapes, sizes, and arrangements, such as an elongated shape, similar to a button hole. Opening 30A may also have woven edges to provide additional strength and resist tearing.

FIG. 28 shows a multi-level implant with three pseudo arthrosis devices 20A-20C coupled to vertebrae 110A-110D. Note that each of pseudo arthrosis devices 20A and 20B has an opening (30A and 30B, respectively) without a grommet. Opening 30A of pseudo arthrosis device 20A overlaps with the upper opening of pseudo arthrosis device 20, whereas opening 30B of pseudo arthrosis device 20B overlaps with the upper opening of pseudo arthrosis device 20C. Openings 30A and 30B lack grommets in order to provide a lower profile for the implant.

Another aspect of the invention relates to artificial replacements for anterior longitudinal ligaments (ALLs). An artificial anterior longitudinal ligament may replace and/or reinforce the function of the patient's natural anterior longitudinal ligament. With the introduction of an interbody implant from an anterior approach for fusion devices or motion-preservation devices, the anterior longitudinal ligament is disrupted. The artificial anterior longitudinal ligament restores the tension band in extension in such a situation. Furthermore, when used with an interbody device (e.g., cores 60 described above), the artificial anterior longitudinal ligament prevent expulsion of an interbody device from the spine. The artificial anterior longitudinal ligament may be used for both fusion and non-fusion applications.

The artificial anterior longitudinal ligament may provide stabilization of other implants and interbody devices. In illustrative embodiments, the implant serves as a tension band to replace the function of the anterior longitudinal ligament and provide mechanical stability. The artificial anterior longitudinal ligament may act as a scaffold for ligamental tissue growth and promote ingrowth of tissue.

The artificial anterior longitudinal ligament is a tension and scaffold band or tab, such as tab 28 of the pseudo arthrosis devices described above, and may be made of similar materials. The band is secured to at least one vertebral body above and below the affected level. It can be used with or without fixation to an interbody device, such as an artificial disc. As an example, tab 28 in the embodiment in FIG. 22 may either be used as a standalone artificial anterior longitudinal ligament, or as an artificial anterior longitudinal ligament coupled to core 60, which serves as an interbody device. The implant could be attached with permanent or resorbable screws, anchors, sutures, bioglue, staples, or with other fastening methods, as desired.

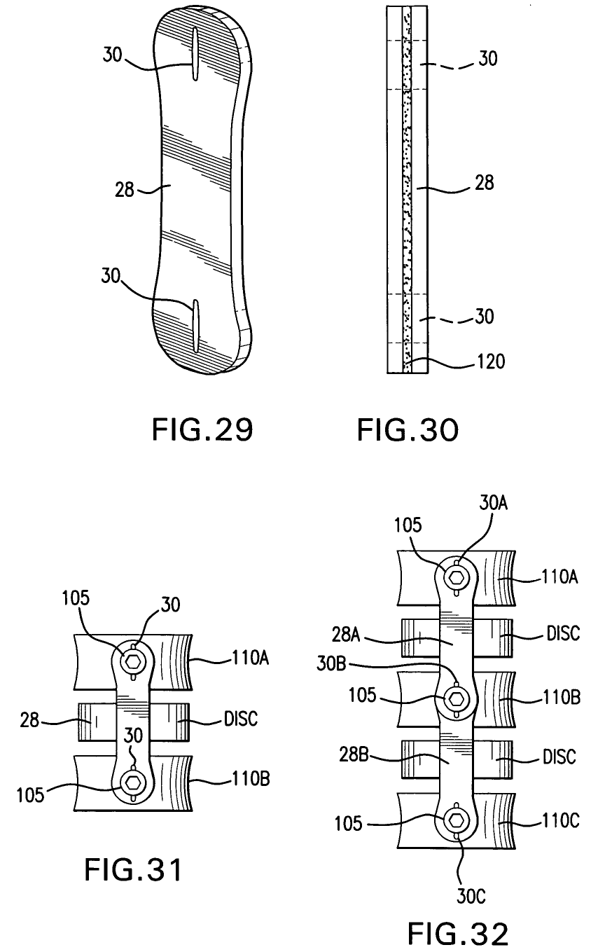

FIG. 29 shows an embodiment of an artificial anterior longitudinal ligament. The artificial anterior longitudinal ligament includes tab 28, having elasticity, with openings 30 for coupling to the vertebrae, as described above. Openings 30 may have a variety of shapes, configurations, and arrangements, as described above.

Tab 28 may include inter-woven elastic material that provides elasticity to it. The tab can therefore limit extension and provide stability to the patient's spine. FIG. 30 shows a side view of an illustrative embodiment of tab 28. Tab 28 may include an inter-woven band 120. Band 120 may be made of any suitable material that provides elasticity to tab 28. Alternatively, tab 28 may be made using elastic materials, or may enclose an elastic band.

FIG. 31 shows an artificial anterior longitudinal ligament coupled to a patient's vertebrae. Tab 28 has two openings 30, each aligned with a respective one of vertebrae 110A and 110B. Screws 105 (or other desired suitable fastener) couples tab 28 to vertebrae 110A, 110B.

Note that, as desired, one may use a plurality of tabs 28 (with the respective openings 30 aligned between neighboring tabs 28) in multi-level applications, as shown, for example, in FIG. 32. Using a suitable fastener (such as screws 105 shown as an example), tab 28A couples to vertebrae 110A and 110B, and tab 28B couples to vertebrae 110B and 110C. Tab 28A has two openings 30A and 30B. Upper opening 30A couples to vertebrae 110A, and lower opening 30B couples to vertebrae 110B. Tab 28B also has two openings, an upper opening (not shown explicitly), and a lower opening 30C. The upper opening of tab 28B aligns with the lower opening 30B of tab 28A and shares a common fastener.

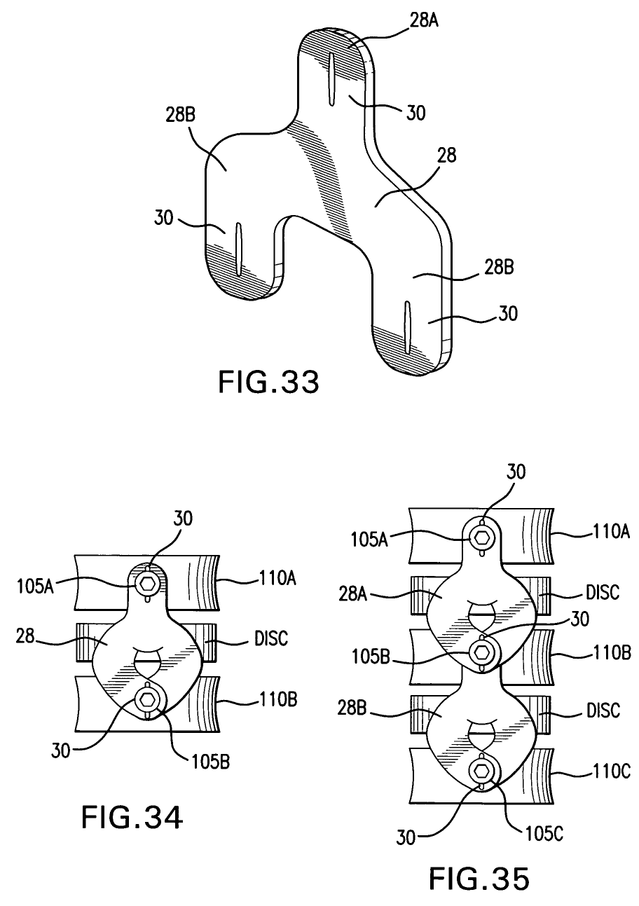

FIG. 33 shows another embodiment of an artificial anterior longitudinal ligament. In this embodiment, tab 28 has an “inverted Y” configuration, with an upper part 28A and a pair of lower parts 28B. Upper part 28A and each of lower parts 28B has an opening 30. Note that the embodiment shown in FIG. 33 may be used as part of a pseudo arthrosis device by coupling an enclosure (including a core) to tab 28, or fabricating the tab integrally to the enclosure, as desired. In this application, the enclosure may include one of the cores 60 described above.

FIG. 34 shows the artificial anterior longitudinal ligament of FIG. 33 implanted in the patient's body. Upper part 28A of tab 28 couples to one vertebra 110A via fastener 105A. Lower parts 28B of tab 28 are arranged in a crossed configuration, such that their respective openings 30 align with one another, and couple to vertebra 110B using fastener 105C.

FIG. 35 shows a multi-level application of the artificial anterior longitudinal ligament of FIG. 33. In this configuration, tab 28A couples to vertebrae 110A and 110B using fasteners 105A and 105B, respectively. The lower parts of tab 28A align with the upper part of tab 28 and may use a common fastener. Thus, tab 28B couples to vertebrae 110B and 110C using fasteners 105B and 105C, respectively.

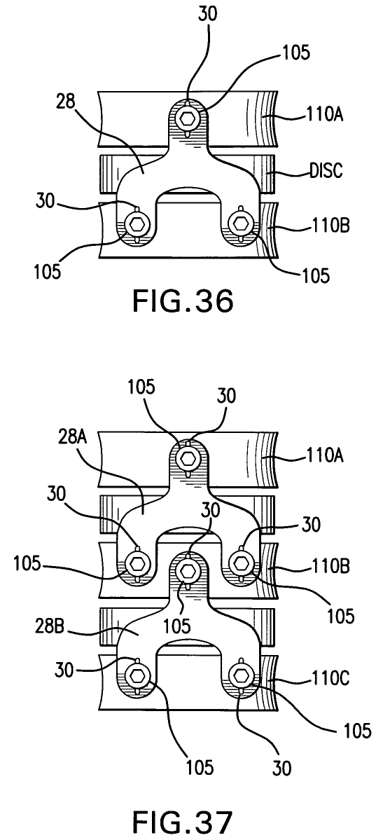

FIGS. 36 and 37 show further applications of the artificial anterior longitudinal ligament of FIG. 33 in single-level and multi-level situations, respectively. Unlike FIGS. 34 and 35, the lower parts of tabs 28 (single-level application in FIG. 36) and tabs 28A and 28B (multi-level application in FIG. 37) are not crossed. Thus, the lower parts of the tabs do not share a common fastener.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

What is claimed is:

1. An implant device, comprising:
an enclosure comprising a core packed within the enclosure, wherein the core comprises a plurality of elongated members; and
at least one tab coupled to the enclosure for attaching the implant device to a vertebra.

2. The device of claim 1, wherein the enclosure comprises a flexible, permeable material.

3. The device of claim 1, wherein the core is flexible.

4. The device of claim 1, wherein the enclosure comprises a top.

5. The device of claim 1, wherein the enclosure comprises a bottom.

6. The device of claim 1, wherein the enclosure comprises a top and a bottom.

7. The device of claim 1, wherein the core further comprises an outer portion surrounding the plurality of elongated members.

8. The device of claim 7, wherein a surface of the outer portion of the core comprises a plurality of indentations.

9. The device of claim 1, wherein the core further comprises a plate, and wherein the plurality of elongated members couple to the plate.

10. An implant device, comprising:
an enclosure comprising a core packed within the enclosure, wherein the core comprises at least one groove on a surface of the core; and
at least one tab coupled to the enclosure for attaching the implant device to a vertebra.

11. The device of claim 10, wherein the enclosure comprises a flexible, permeable material.

12. The device of claim 10, wherein the core is flexible.

13. The device of claim 10, wherein the at least one groove is disposed laterally on the surface of the core.

14. The device of claim 10, wherein the at least one groove is disposed on the surface of the core in an anterior-to-posterior direction.

15. The device of claim 10, wherein the at least one groove comprises a spiral.

16. The device of claim wherein the at least one groove comprises a plurality of co-centric circles.

17. An artificial ligament implant, comprising a tab having elasticity, wherein the tab comprises first and second openings configured to couple the tab to first and second vertebrae as an artificial longitudinal ligament.

18. The artificial ligament implant of claim 17, wherein the tab comprises inter-woven elastic material.

19. The artificial ligament implant of claim 17, wherein the tab comprises an elastic band.

20. The artificial ligament implant of claim 17, wherein the tab further couples to an artificial disc disposed between the first and second vertebrae.
